(19) United States
(12) Patent Application Publication
(10) Pub. No.: US 2010/0286600 A1
(43) Pub. Date: Nov. 11, 2010

(54) TRANSDERMAL PATCH DEVICE

(76) Inventor: D. Bommi Bommannan, Los Altos, CA (US)

Correspondence Address: D. BOMMI BOMMANNAN, 2251 Grant Road, Suite B, LOS ALTOS, CA, 94024, US

(21) Appl. No.: 12/777,037

(22) Filed: May 10, 2010

Related U.S. Application Data
(60) Provisional application No. 61/176,819, filed on May 8, 2009.

(51) Int. Cl. A61M 5/168; A61K 31/4402

(52) U.S. Cl. 604/66; 514/357

(57) ABSTRACT

A patch device worn by a user is disclosed that is configured to sense the presence of a harmful substance in the environment and to deliver an antidote to that substance into the user without the intervention of the user. The patch device incorporates a sensing mechanism configured to sense a presence of the harmful substance in the environment surrounding the patch device. On sensing the presence of the harmful substance, the sensor triggers a delivery mechanism, which causes the therapeutic agent to be injected into the user. The therapeutic agent acts to antidote the effects of the harmful substance within the user. The patch device could be used for protection against exposure to substances such as nerve gas and pesticides. The patch could be used to deliver the therapeutic agent either subcutaneously or transdermally.

### CROSS-REFERENCE TO RELATED APPLICATION

This application claims the benefit of U.S. Provisional Application No. 61/176,819, filed May 8, 2009. The entire teachings of the above application are incorporated herein by reference.

### FIELD OF THE INVENTION

This invention relates to automated patch devices for therapeutic purposes.

### DESCRIPTION OF THE RELATED ART

Automatic injection devices are used to deliver therapeutic agents for both acute and long term treatment of disease conditions. Long term treatment for conditions such as diabetes involves monitoring within the patient the concentration of blood glucose at suitable intervals and administering insulin so that the concentration of blood glucose is maintained within a desired band. Here injection of the agent, in this case insulin, is triggered by the value of blood glucose measured within the patient. For many years injection devices have been available that require inputs from the user to adjust the dose of the injected agent. However, the major drivers for development of improved devices have been automatic monitoring and delivery as opposed to measurement and injection by conscious intervention of the user.

The need for automated delivery of medications is even greater in the case of acute conditions such as heart attack or anaphylactic shock. In such cases, the desired medication must be quickly infused when the need arises. For this purpose, the device can either be placed in skin contact by the user to deliver the dose, or it can be strapped or affixed with adhesive to be worn by the user. Of the first category, injection pen devices have been available for a number of years as in U.S. Pat. No. 6,517,517, which discloses a cylindrically shaped injection device operated by a spring-driven plunger that is placed in contact with the skin for injecting the medication. Another such device with a flat shape is described in U.S. Pat. No. 6,979,316, which incorporates safety features such as markings on the housing to serve as operational guide, visibility in the dark and other design features intended to prevent accidental triggering of the injection. However, such devices have the drawback that they have to be carried separately at all times and must be correctly activated by the user in the rare event of need.

A particular situation where acute treatment is needed is for delivering an antidote where exposure to toxins or harmful substances such as nerve gas is involved, as encountered by soldiers or other support personnel in combat. The use of atropine injections for nerve gas exposure is known and autoinjector devices have been developed that can be used by soldiers or other technical personnel working in hazardous environments as in U.S. Pat. No. 5,092,843, for example. However, such devices must be activated by the user within a short time after exposure, i.e., before the user is incapacitated. More recently, a wearable device has been disclosed in U.S. Patent application No. 2005/0182307 that uses a sophisticated sensor, diagnostic and feedback system to actuate injection of an antidote on detection of a harmful substance in the environment. However, the device uses sensing of blood and other fluids in the user's skin and is complicated and expensive.

From the ongoing discussion, it is clear that the available devices for treating exposure to harmful substances have various drawbacks, including the requirement of user intervention. It is therefore an object of this invention to provide a simple and inexpensive device that can be worn by a person subjected to exposure to a harmful substance, wherein detection of the substance and injection of the antidote do not involve a voluntary action by the person.

### SUMMARY OF THE INVENTION

The invention is a patch device worn by a user to treat exposure to harmful substances. In one embodiment the patch device comprises a sensor for detecting presence of a harmful substance in the environment surrounding the user, a therapeutic agent reservoir or depot, and a delivery mechanism. On detecting the presence of the harmful substance, the sensor triggers delivery of the therapeutic agent into the user. The therapeutic agent then antidotes the harmful substance within the user.

In one embodiment the therapeutic agent is present in liquid form and is delivered subcutaneously. The therapeutic agent is delivered using spring force, gas pressure, electrochemical force or force due to phase transformation of a shape memory alloy. The sensor can be an electrochemical cell or a chemical substance.

In another embodiment the therapeutic agent is present in an adhesive matrix and is delivered transdermally. The matrix incorporates a chemical substance that reacts with the harmful substance to produce an exothermic reaction, thereby releasing the therapeutic agent for transdermal delivery.

The patch device of the present invention is attached to the skin of the patient using an adhesive patch or it is strapped on to the user's skin.

### BRIEF DESCRIPTION OF THE DRAWINGS

The invention has other advantages and features which will be more readily apparent from the following detailed description of the invention and the appended claims, when taken in conjunction with the accompanying drawings, in which:

FIG. 1 illustrates schematically the treatment concept.

FIGS. 2A-2C show an embodiment in which spring force is used to inject drug from a reservoir.

FIGS. 3A-3C show an embodiment that uses gas pressure to inject drug from a reservoir.

FIGS. 4A-4B show an embodiment comprising an adhesive patch.

### DETAILED DESCRIPTION

Although the detailed description contains many specifics, these should not be construed as limiting the scope of the invention but merely as illustrating different examples and aspects of the invention. It should be appreciated that the scope of the invention includes other embodiments not discussed herein. Various other modifications, changes and variations which will be apparent to those skilled in the art may be made in the arrangement, operation and details of the method and apparatus of the present invention disclosed herein without departing from the spirit and scope of the invention as described here.

The invention is a device containing a therapeutic agent that is worn by a user for treatment against exposure to a harmful substance or analyte. The patch device comprises a sensor that detects an analyte in the environment that is capable of adversely affecting the user. On detecting the analyte, the patch device delivers the therapeutic agent, automatically and without user intervention, into the user's body. The therapeutic agent then counteracts the effects of the analyte on the user.

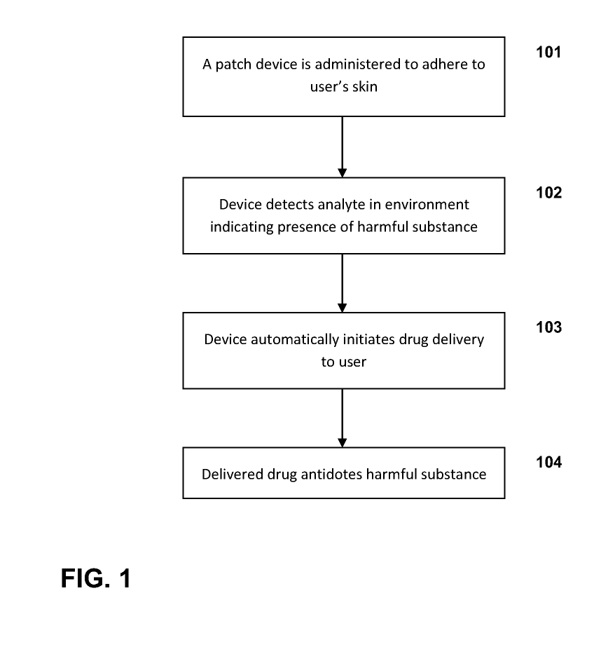

A flow diagram illustrating a method for automatic analyte detection and therapeutic agent delivery to a user, without the need for user interaction, is shown in FIG. 1. At step 101, a wearable patch device is attached to the skin of a user. The device is configured to detect the presence of a harmful substance in the surrounding environment. In one embodiment, the device is configured to detect a harmful substance in the ambient atmosphere surrounding the user. In another embodiment, the device is configured to detect an analyte that is present in the user's body. At step 102, the harmful substance is detected, which causes the device to automatically initiate therapeutic agent delivery to the user at step 103. The delivery initiation at step 103 occurs automatically and without intervention of the user. In step 104, the therapeutic agent antidotes the effects of the harmful substance within the user's system.

The present embodiments may be configured to detect various substances that may be harmful to the wearer of the device and to deliver the therapeutic agent at a sufficient quantity or concentration to antidote the harmful substance to substantially remedy or avoid bodily harm to the wearer. Additionally and optionally, the present embodiments may deliver the therapeutic agent at a sufficient quantity or concentration such that sufficient time is available for the wearer to seek further medical attention before substantial bodily harm occurs.

In another embodiment, the device, upon sensing a harmful substance can alert the wearer of the presence of the harmful substance. The alert could be in the form of a visual (blinking LED), auditory (high pitched beeps) or tactile signal (vibration). The user can then decide to initiate the drug delivery by pressing a button or the like on the device.

In one embodiment, the harmful substance is an allergen that causes allergic symptoms in the user and the therapeutic agent delivered to the user is an anti-allergy agent. The anti-allergy agent may be an antagonistic agent that blocks the action of the allergen, or prevent activation of cells and degranulation processes. Such therapeutic agent may include antihistamines, cortisone, dexamethasone, hydrocortisone, epinephrine, theophylline and cromolyn sodium. Additionally, the therapeutic agent may be anti-leukotrienes, such as montelukast or zafirlukast, or any other anti-allergy agent.

In another embodiment, the harmful substance is a toxin such as cyanide, anthrax, organophosphates, various neurotoxins, pesticide sprays such as chlorpyrifos or other biologically harmful analytes, and the therapeutic agent is one or more antidotes to the toxin such as hydroxycobalamin, atropine, anti-cholinergic agent, pralidoxime chloride, various antibiotics, such as ciprofloxacin, doxycycline, erythromycin, vancomycin or penicillin, or other antidotes that is configured to remedy the effect of one or more toxins.

In yet another embodiment, the therapeutic agent is a preventive agent such that upon detection of the harmful substance in the ambient environment, the therapeutic agent is administered to the user to substantially prevent any potential harm to the user from potential exposures to the harmful substance.

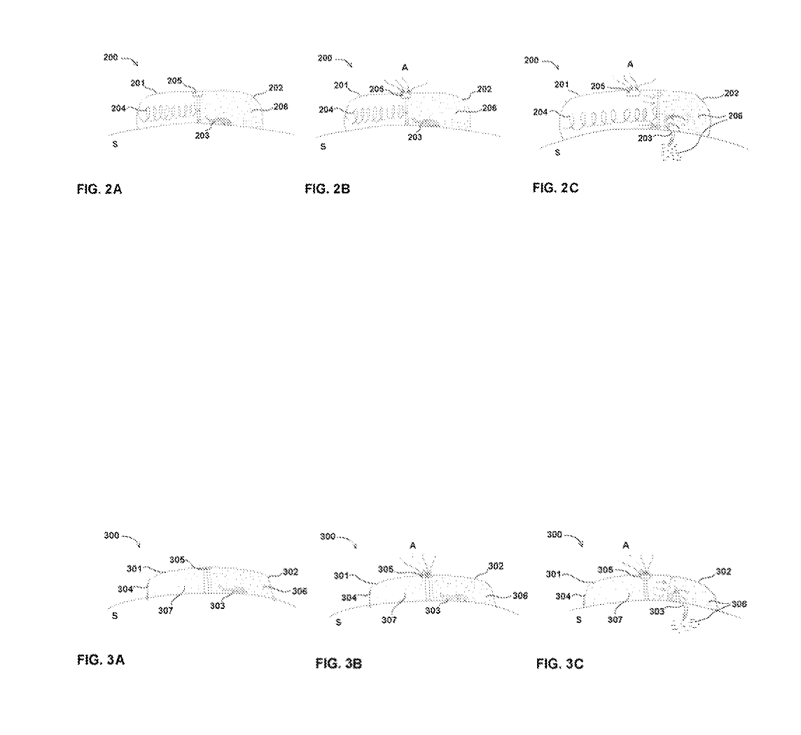

Further embodiments of the invention generally outlined above are described in particular in the following description and are exemplarily shown in FIGS. 2A-2C and 3A-3C. Particularly, FIGS. 2A-2C and 3A-3C illustrate embodiments of the present invention wherein the therapeutic agent is present in liquid form. FIGS. 2A-2C illustrate a patch device in which the liquid therapeutic agent is propelled using a spring. The patch device 200 is placed on the surface of the user's skin S. The device comprises a housing 201, which is made of a suitable backing material, and comprises at least one reservoir 202, needle 203, spring 204, and sensor 205, wherein the reservoir 202 contains the therapeutic agent 206 in a liquid form. Suitable backing materials include metal foils such as aluminum foil, polyesters such as polyethylene terephthalate, polyamides such as polycaprolactones, polyolefins such as polyethylene or polypropylene, polyacrylates such as polymethylmethacrylate or acrylamide, polyurethanes, vinyl polymers and copolymers such as polyvinyl chloride or polyvinylacetate, polyurethanes, cellophanes or other similar materials. Multi-layer laminates may also be used. A particularly preferred backing material comprises a skin-colored polyester film/metal foil laminate.

As shown in FIG. 2B, on detection of the harmful substance or analyte A by the sensor 205, the spring 204 is released to act against the reservoir 202, causing the hollow needle 203 to pierce the skin S and to deliver the therapeutic agent 206. Compression of the reservoir 202 by the spring 204 causes therapeutic agent 206 to be injected subcutaneously through the needle 203 into the user, as shown in FIG. 2C. The therapeutic agent 206 then antidotes the effects of the harmful substance within the user.

Upon detection of a harmful substance, the spring can be activated using one of many methods that are known to one skilled in the art. For example, the sensor can be an electrochemical sensor that can generate an electrical signal upon detecting the harmful signal. The sensor is operatively coupled to the spring using an electromagnetic switch, which is activated by the electrical signal generated by the sensor. In another embodiment, compression of the reservoir to achieve drug delivery can be accomplished using a shape memory alloy, such as Nitinol, that is in contact with the reservoir. Shape memory alloys can be expanded from a compressed state upon exposure to heat. By appropriate selection of a sensor material, the sensor can react with the harmful substance (analyte) and provides an exothermic response. The heat that is generated can then expand the shape memory alloy that is in contact with the reservoir.

Another embodiment of the patch device with the therapeutic agent in a liquid form is shown in FIGS. 3A to 3C, in which the patch is activated using gas pressure. As shown in FIG. 3A, the patch device 300 is worn by placing it on the surface of the user's skin S. The device housing 301 contains at least one reservoir 302, needle 303, gas reservoir 304, and sensor 305. The reservoir 302 contains the therapeutic agent 306 in liquid form, while the gas reservoir 304 holds gas under pressure.

Specifically, the gas reservoir 304 comprises a holding element that is in communication with the reservoir 302. The holding element is configured to hold a gas 307 under sufficient pressure such that upon release of the gas 307 from the holding element, the gas is configured to pressurize the reservoir 304. Optionally, the gas reservoir 304 may comprise a compression element such as a spring or a micromotor that is configured to pressurize and maintain the gas 307 at a sufficient pressure. The optional compression element may also aid in the release of the gas 307 to pressurize the reservoir 304.

As shown in FIG. 3B, when the user is exposed to the harmful substance or analyte A, it is detected by the sensor 305. The sensor 305 then communicate a signal that causes the gas 307 that is kept under pressure in the gas reservoir 304, to be released, wherein the gas 307 then pressurizes the reservoir 302. As further shown in FIG. 3C, pressurizing the reservoir 302 causes the needle 303 comprising a lumen to pierce the skin S, and the therapeutic agent 306 is injected subcutaneously into the user therethrough. The therapeutic agent 306 then substantially antidotes the effects of the harmful substance within the user.

The embodiments described above may optionally comprise at least one transducer, at least one processor, and at least one memory such as electrical erasable programmable memory (EPROM), ROM, RAM, etc. The signal detected by the sensor may be embedded with information such as the amount of harmful substance detected in the environment, distance of the harmful substance from the user, etc. The transducer is configured to transmit a signal received from the sensor to the processor. The processor is then configured to determine and control the appropriate amount of therapeutic agent to be delivered to the user based on the signal from the sensor and a dosing parameter stored in the memory. In such embodiment, the device may be configured to deliver the appropriate amount of therapeutic agent to antidote the amount of the harmful substance detected by the sensor to avoid over- or under-dosing.

In another embodiment, the device may be configured to detect and antidote multiple types of harmful substances. The device may comprise multiple sensors, multiple reservoirs, at least one transducer, at least one processor, at least one memory such as an EPROM, and a delivery mechanism such as the spring or gas pressure mechanisms described above. The multiple sensors are configured such that at least one of the sensors is configured to detect a harmful substance different from that detected by at least one other sensor. The reservoirs are configured to store multiple therapeutic agents to counteract the different types of harmful substances that are detectable by the sensors.

Upon detection of a harmful substance, the transducer is configured to transmit a signal received from the sensor to the processor. The processor then determines the type of harmful substance detected and activates the delivery of the appropriate therapeutic agent configured to counteract the detected harmful substance. Furthermore, as described above, the processor may be configured to select the appropriate amount of therapeutic agent to be delivered to the user based on the amount of harmful substance detected by the sensor and the dosing parameter stored in the memory.

The devices in the above embodiments have been illustrated to operate using spring force and gas pressure as delivery mechanisms. It is envisioned that other ways of effecting delivery of the therapeutic agent include use of electrochemical force, shape memory elements, micromotor, and other mechanisms may be used as the delivery mechanism.

The sensing mechanism used in the above embodiments may comprise an electrochemical cell, a chemical reaction, or other sensing mechanism that is configured to determine the presence of the harmful substance or analyte in the environment of the user. The sensing mechanism may be calibrated to various degrees of sensitivity to detect various quantities or concentrations of the harmful substance. In one embodiment, the sensing mechanism is configured to transmit a binary signal to a delivery mechanism or a processor to signify whether a harmful substance is present or not. In another embodiment, the sensing mechanism is configured to transmit a signal with additional information, such as amount of substance, strength of the substance, distance of the substance from the user, etc.

A variety of materials can be used to construct the patch devices of the above embodiments. The device housing 201 or 301 can be made of any rigid and durable plastic such as polyethylene, polypropylene, ABS or polyurethane. The flexible reservoir can be made of a suitable grade of polyethylene. The needle can be made of surgical stainless steel or other biocompatible material. Furthermore, the housing 201 or 301 may comprise one or more sealing O-rings to enable the device to be water-proof or water-resistant to minimize damage from exposure to the elements or moistures produced by the body.

In one embodiment, the device is configured as a wearable patch or a band that is affixed to the skin of the user using a biocompatible adhesive. In another embodiment, the device comprises one or more straps such that the device may be strapped on to the user, for example the device may be strapped across the chest of the user. It is further contemplated that the device may be incorporated in to a piece of clothing, gear or accessory. For example, the device may be incorporated in to a watch, bracelet, bangle, helmet, straps of a backpack, holster, etc.

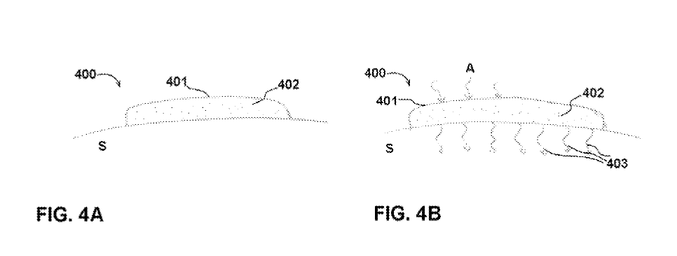

Another embodiment of the device in the form of a transdermal delivery patch is shown in FIGS. 4A and 4B. As shown in FIG. 4A, patch device 400 is placed on the skin S of the user when worn. Patch device 400 comprises a matrix 401 that contains a mixture of adhesive 402 and a therapeutic agent 403. Matrix 401 further comprises a sensing agent (not shown) that is responsive to analyte A. On exposure to the harmful substance or analyte A, the sensing agent causes the therapeutic agent 403 to be released transdermally into the user, as shown in FIG. 4B. Therapeutic agent 403 then antidotes the effects of the harmful substance A within the user.

In one embodiment the sensing element is a chemical substance that produces an exothermic response to the harmful substance. The heat generated by the sensing element causes the therapeutic agent 403 to be delivered transdermally into the user.

The advantage of such a system is that the activation of the transdermal patch delivery system does not depend on any voluntary action by the subject, but delivery of the antidote happens automatically once the sensor detects the harmful substance.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A patch device for treatment of a user, comprising:
a sensing mechanism configured to sense presence of an analyte in an environment surrounding the user;
a reservoir configured to store a therapeutic agent which counteracts the effects of the analyte; and
a delivery mechanism configured to deliver the therapeutic agent to the user;
wherein the sensing mechanism is further configured to activate the delivery mechanism upon sensing the presence of the analyte, thereby delivering the therapeutic agent to the user in order to counteract the effects of the analyte within the user.

2. The patch device of claim 1, wherein the therapeutic agent is delivered subcutaneously.

3. The patch device of claim 1, wherein the therapeutic agent is delivered transdermally.

4. The patch device of claim 1, wherein the analyte comprises an organophosphate agent.

5. The patch device of claim 1, wherein the therapeutic agent comprises an anti-cholinergic agent or pralidoxime chloride.

6. The patch device of claim 1, wherein the therapeutic agent comprises an anti-allergy agent.

7. The patch device of claim 2, wherein the delivery mechanism is driven by spring force.

8. The patch device of claim 2, wherein the delivery mechanism is driven by gas pressure.

9. The patch device of claim 2, wherein the delivery mechanism is driven by an electrochemical reaction.

10. The patch device of claim 2, wherein the delivery mechanism is driven by a shape memory element.

11. The patch device of claim 1, wherein the sensing mechanism comprises an electrochemical cell.

12. The patch device of claim 1, wherein the sensing mechanism comprises a chemical reaction.

13. The patch device of claim 1, wherein the device is stuck to the skin using an adhesive.

14. The patch device of claim 1, wherein the device is strapped on to the skin of the user.

15. The device of claim 3, wherein the sensing mechanism comprises a chemical change.

16. The device of claim 3, wherein the activation mechanism comprises a chemical change that releases a therapeutic agent.

17. The device of claims 15-16, wherein the sensing and activation steps are the same.

18. A method of treatment of a user comprising:
sensing an analyte in the ambience surrounding a user; and
delivering a therapeutic agent into the user when the analyte is detected;
wherein the delivering is configured to proceed without the intervention of the user.

19. The method of claim 18, wherein the analyte comprises an organophosphate nerve agent.
